(19) United States
(12) Patent Application Publication
(10) Pub. No.: US 2005/0186234 A1
(43) Pub. Date: Aug. 25, 2005

(54) Pack-a-poof

(76) Inventor: Colleen Kelly, Export, PA (US)

Correspondence Address: JAMES RAY & ASSOCIATES, 2640 PITCAIRN ROAD, MONROEVILLE, PA, 15146, US

(21) Appl. No.: 11/018,461

(22) Filed: Dec. 21, 2004

Related U.S. Application Data
(60) Provisional application No. 60/546,035, filed on Feb. 19, 2004.

(51) Int. Cl. A61K 7/50

(52) U.S. Cl. 424/401

(57) ABSTRACT

A disposable apparatus for body cleansing, the disposable apparatus comprises a plurality of layers of net like material bunched together in a predetermined shape forming a poof. There is a means for securing the plurality of layers of the net like material that is bunched together to form the poof and at least one gelatinous capsule having at least one of a liquid soap and a shower gel encapsulated therein disposed within and retained by the net like material. The gelatinous capsule having at least one of liquid soap and shower gel encapsulated therein has a predetermined shape.

### CROSS REFERENCE TO RELATED APPLICATIONS

This application is closely related to and claims benefit from U.S. Provisional Application Ser. No. 60/546,035 filed Feb. 19, 2004.

### FIELD OF THE INVENTION

The present invention relates, in general, to a cleansing apparatus, and, more particularly, the present invention relates to a disposable cleansing apparatus made of a polymer netting which includes a gelatinous capsule containing liquid or gel type soaps encapsulated therein.

### BACKGROUND OF THE INVENTION

In recent years, there has been a major change in American's shower products. The puff (or “poof”) has been used as a cleansing agent as a shower tool for many consumers. It has become an indispensable tool for many because of the advantages it posses over traditional bar soaps. The puff provides a way of washing with less mess in the shower, gentle exfoliation and more soap suds than traditional soap. However, the puff does have drawbacks. First and foremost, it does not travel well. When it is packed with a bottle of shower gel, it becomes rather bulky. Further, the puff becomes saturated with water during use and, thus, it must be placed in a plastic bag after use before being packed in the suitcase. Being wet, it could also be a breeding ground for bacteria. Another alternative is to simply discard the puff; however, with presently available puffs this may not be economically feasible. Thus, there is a need for an improved puff or poof which can eliminate the drawbacks found with present poofs.

### SUMMARY OF THE INVENTION

In a first aspect the present invention provides a disposable apparatus for body cleansing, the disposable apparatus comprises a plurality of layers of a flexible material bunched together in a predetermined shape forming a poof. There is a means engageable with said poof for securing the plurality of layers of the net like material together and at least one capsule having a predetermined solubility and having at least one of a liquid soap and a shower gel encapsulated therein disposed within and retained by the net like material. The capsule having at least one of liquid soap and shower gel encapsulated therein has a predetermined shape.

In another aspect of the invention there is provided a disposable package for body cleansing. The disposable package includes a disposable apparatus for body cleansing, the disposable apparatus comprises a plurality of layers of flexible material bunched together in a predetermined shape forming a poof. There is a means engageable with the poof for securing the plurality of layers of the net like material together and at least one capsule having a predetermined solubility and having at least one of a liquid soap and a shower gel encapsulated therein disposed within and retained by the flexible material. The capsule having at least one of liquid soap and shower gel encapsulated therein has a predetermined shape. The apparatus further includes a container for containing such disposable apparatus for body cleansing.

In yet another embodiment of the invention there is provided a disposable apparatus for surgical cleansing, said disposable apparatus comprises a plurality of layers of flexible material bunched together in a predetermined shape forming a poof. There is a means engageable with the poof for securing the plurality of layers of such net like material together, and at least one capsule having a predetermined solubility and having at least one of a liquid medicated soap and a medicated gel encapsulated therein disposed within and retained by such net like material. The capsule of the at least one of such liquid medicated soap and such medicated gel encapsulated therein having a predetermined shape.

### OBJECTS OF THE INVENTION

It is, therefore, one of the primary objects of the present invention to provide a disposable “poof” which contains a capsule of liquid soap or shower gel.

Another object of the present invention is to provide a disposable poof with a gelatin material to form the capsule with which to enclose the liquid soap or shower gel.

Yet, another object of the present invention is to provide a disposable poof which uses a polymer netting which is relatively inexpensive.

Still another object of the present invention is to provide a disposable poof which is convenient and travel-sized.

Another object of the present invention is to provide a disposable poof which is smaller than traditional poofs.

It is another object of the present invention to provide a disposable poof in which the gelatinous capsule will dissolve when it becomes wet to release the shower gel or liquid soap.

It is still another object of the present invention to provide a disposable cleansing apparatus which contains medic soap or gel for surgical cleansing.

In addition to the various objects and advantages of the invention which have been described in some specific detail above it should be noted that various other objects and advantages of the present invention will become more readily apparent to those persons who are skilled in the relevant art from the following more detailed description, particularly, when such description is taken in conjunction with the attached drawing Figures and with the appended claims.

### BRIEF DESCRIPTION OF THE DRAWINGS

FIG. 1 is a partial perspective view of the disposable apparatus according to an embodiment of the invention packed in a container.

FIG. 2 is a partial perspective view of the disposable apparatus showing the encapsulated shower gel or liquid soap.

FIG. 3 is a partial perspective view of the apparatus according to an alternate embodiment of the invention.

FIG. 4 is a partial perspective view of the disposable apparatus showing the thread used for securing the net like material.

### BRIEF DESCRIPTION OF THE PRESENTLY PREFERRED AND VARIOUS ALTERNATIVE EMBODIMENTS OF THE INVENTION

Prior to proceeding to the more detailed description of the invention, it should be noted that identical components having identical functions have been designated with identical reference numerals throughout the several views illustrated in the drawings for the sake of clarity.

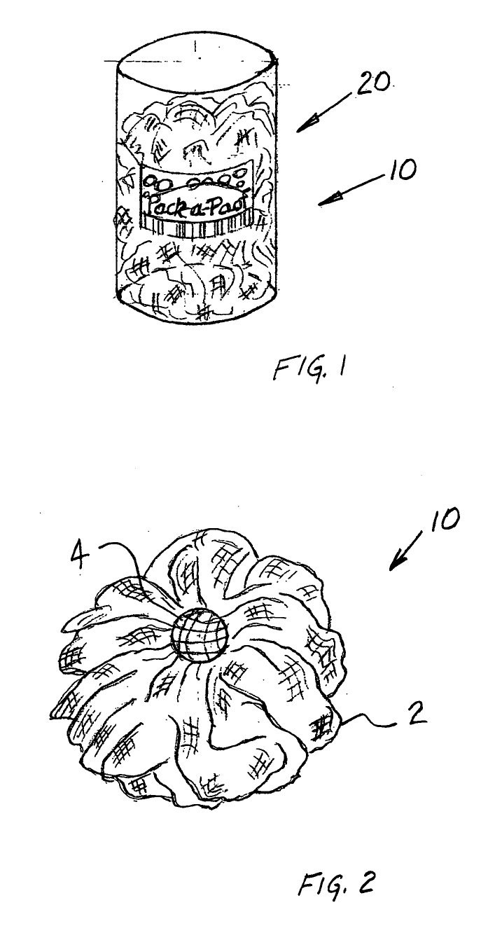

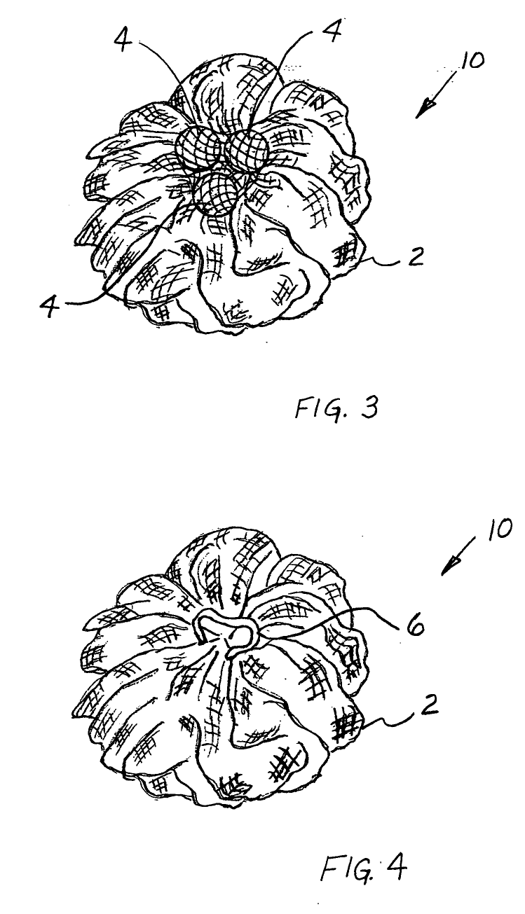

Now reference is made, more particularly, to drawing FIGS. 2 and 3. Illustrated therein is a disposable apparatus called a poof, generally designated 10, for body cleansing. Traditional poofs 10 or puffs are used to cleanse the body, and provide the benefits of gentle exfoliation and robust soap suds. The poofs 10 provide a distinct advantage over washing with conventional bar soaps or even with shower gels. Pouring a shower gel directly into ones hand isn't effective and much of the gel is lost by sliding off the hand.

The “poof” 10 of the present invention is a sponge like object constructed of a flexible material 2. It is preferred that such flexible material is a net like material and even more preferred that such net like material is a polymer netting 2. Such poof 10 contains a plurality of layers of such polymer netting 2 bunched together in a predetermined shape to form such poof 10.

There is further a means 6 engageable with such poof 10 for securing such plurality of layers of the net like material 2. It is presently preferred that such means for securing the net like material 2 be one of thread 6 as is seen in FIG. 4. Such thread 6 is tied together. It is also presently preferred that the predetermined shape of the poof 10 is substantially spherical. The poof 10 includes a capsule 4 having a predetermined solubility and having a shower gel or a liquid soap encapsulated therein. It is also presently preferred that such capsule 4 be made of a gelatinous material which will dissolve upon contact with water. Thus, the poof 10 contains all that is necessary for cleansing in a shower or bath. The gelatinous capsule 4 retains the shower gel or liquid soap until the user is ready to use it. Merely wetting the poof 10 which will also wet the gelatinous capsule 4 is all that is necessary to dissolve the gelatinous capsule 4 and release the shower gel or soap enclosed therein. The gelatinous capsule 4 with the gel or soap enclosed therein has a predetermined shape. In a presently preferred embodiment of the invention such shape is substantially spherical; however, it is within the scope of the invention that such predetermined shape could be virtually any shape that would be desired.

In an alternate embodiment of the invention the apparatus includes more than one gelatinous capsule 4 with the shower gel or liquid soap enclosed therein. As seen in FIG. 3 a poof 10 is shown wherein there are three gelatinous capsules 4 with soap inside. In the case where more than one gelatinous capsule 4 are included in the poof 10 the thickness of the gelatinous capsule 4 enclosing the shower gel would be different for each capsule so that the gelatinous capsule 4 would dissolve at different times so as to release the second shower gel after the first shower gel has already been released and possibly used up.

In still another alternate embodiment of the invention there is provided a disposable package, generally designated 20, for containing the disposable poof 10. The disposable poof 10 would be the same as that described above. It is presently preferred that such disposable package 20 is a plastic bag.

Yet in another embodiment of the invention there is provided a disposable cleansing apparatus 10 similar to the poof 10 described previously except this embodiment is designed for use in surgical scrubbing. The basic difference in this cleansing apparatus 10 is that the gelatinous capsule 4 contains a medicated liquid soap or a medicated gel for such surgical cleansing. It should be noted that such medicated liquid soap and medicated gel includes anti-microbial, anti-bacterial, povidone-iodine (betadine), antiseptic, surgical soap, and sterilizing soap.

Thus, the present invention provides a disposable poof 10 with a shower gel or liquid soap encapsulated within a gelatinous capsule 4 for use as a cleansing agent and then is thrown away. Such disposable poof 10 is carried in a plastic bag 20 which is also disposable. Since the poof 10 is disposable the problem with packing a wet poof is eliminated since the poof 10 is just discarded after use. Also the danger of the wet stored poof being a breeding ground for bacteria is also eliminated since there is no storage of wet poofs 10.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

1. A disposable apparatus for body cleansing, said disposable apparatus comprising:
(a) a plurality of layers of flexible material bunched together in a predetermined shape forming a poof;
(b) a means engageable with said poof for securing said plurality of layers of said net like material together; and
(c) at least one capsule having a predetermined solubility and having at least one of a liquid soap and a shower gel encapsulated therein disposed within and retained by said flexible material, said capsule of said at least one of said liquid soap and said shower gel encapsulated therein having a predetermined shape.

2. The apparatus for body cleansing, according to claim 1, wherein said flexible material is a net like material.

3. The apparatus for body cleansing, according to claim 2, wherein said net like material is an organic polymer netting.

4. The apparatus for body cleansing, according to claim 1, wherein said means for securing said plurality of layers of said net like material in said form of said poof is thread.

5. The apparatus for body cleansing, according to claim 4, wherein said thread is a synthetic fabric.

6. The apparatus for body cleansing, according to claim 5, wherein said synthetic fabric is nylon.

7. The apparatus for body cleansing, according to claim 1, wherein said capsule having a predetermined solubility is gelatinous.

8. The apparatus for body cleansing, according to claim 7, wherein said at least one gelatinous capsule of said at least one of said liquid soap and said shower gel encapsulated therein is a plurality of gelatinous capsules of said at least one of said liquid soap and said shower gel encapsulated therein.

9. The apparatus for body cleansing, according to claim 7, wherein said at least one of said liquid soap and said shower gel encapsulated in said at least one gelatinous capsule is released from said gelatinous capsule by wetting said gelatinous capsule.

10. The apparatus for body cleansing, according to claim 8, wherein a thickness of said gelatinous capsule in said plurality of gelatinous capsules is different for each gelatinous capsule so that said gelatinous capsule will dissolve at different times thereby releasing such at least one of liquid soap and shower gel encapsulated therein at different times.

11. The apparatus for body cleansing, according to claim 1, wherein said predetermined shape of said capsule containing said at least one of a liquid soap and said shower gel is substantially spherical.

12. The apparatus for body cleansing, according to claim 1, wherein said predetermined shape of said plurality of layers of said flexible material forming said poof is substantially spherical.

13. A disposable package for body cleansing, said disposable package comprising:
(a) a disposable apparatus for body cleansing, said disposable apparatus including:
(i) a plurality of layers of flexible material bunched together in a predetermined shape forming a poof;
(ii) a means engageable with said poof for securing said plurality of layers of said flexible material together;
(iii) at least one capsule having a predetermined solubility and having at least one of a liquid soap and a shower gel encapsulated therein disposed within and retained by said flexible material, said capsule of said at least one of said liquid soap and said shower gel encapsulated therein having a predetermined shape; and
(b) a container for containing said disposable apparatus for body cleansing.

14. The package, according to claim 13, wherein said container is a plastic bag.

15. The apparatus for body cleansing, according to claim 13, wherein said means for securing said plurality of layers of said flexible material in said form of said poof is thread.

16. The apparatus for body cleansing, according to claim 15, wherein said thread is a synthetic fabric.

17. The apparatus, according to claim 13, wherein said predetermined shape of said capsule containing said at least one of a liquid soap and said shower gel is substantially spherical.

18. The apparatus for body cleansing, according to claim 13, wherein said predetermined shape of said of said plurality of layers of said flexible material forming said poof is substantially spherical.

19. A disposable apparatus for surgical cleansing, said disposable apparatus comprising:
(a) a plurality of layers of flexible material bunched together in a predetermined shape forming a poof;
(b) a means engageable with said poof for securing said plurality of layers of said flexible material together; and
(c) at least one capsule having a predetermined solubility and having at least one of a liquid medicated soap and a medicated gel encapsulated therein disposed within and retained by said flexible material, said capsule of said at least one of said liquid medicated soap and said medicated gel encapsulated therein having a predetermined shape.

20. The apparatus, according to claim 19, wherein said at least one of a liquid medicated soap and a medicated gel are selected from the group consisting of anti-microbial, anti-bacterial, povidone-iodine, antiseptic, surgical soap, and sterilizing soap.
